(19) United States
(12) Patent Application Publication
(10) Pub. No.: US 2005/0142065 A1
(43) Pub. Date: Jun. 30, 2005

(54) Screening method

(76) Inventors: Koki Kato, Kobe-shi (JP); Yasutaka Nagisa, Nara-shi (JP); Hiroyuki Odaka, Kobe-shi (JP)

Correspondence Address: TAKEDA PHARMACEUTICALS NORTH AMERICA, INC;INTELLECTUAL PROPERTY DEPARTMENT, 475 HALF DAY ROAD, SUITE 500, LINCOLNSHIRE, IL, 60069, US

(21) Appl. No.: 10/512,237

(22) PCT Filed: Apr. 24, 2003
(86) PCT No.: PCT/JP03/05254

(30) Foreign Application Priority Data
Apr. 26, 2002 (JP) 2002125469

(51) Int. Cl. A61K 49/00; A01K 67/027

(52) U.S. Cl. 424/9.2; 800/14

(57) ABSTRACT

The present invention provides a method of screening for an agent for the treatment of human diabetes mellitus, which comprises administering a test compound to a diabetes mellitus model non-human mammal, and selecting a compound that causes a decrease of not less than 0.5% in a glycated hemoglobin value after administration.

### TECHNICAL FIELD

The present invention relates to a method of screening for an agent for the treatment of human diabetes mellitus.

### BACKGROUND ART

Glycated hemoglobin (hereinafter sometimes to be abbreviated as GHb) is also called glycosylated hemoglobin or glycohemoglobin, and is a hemoglobin bonded with sugar such as D-glucose, glucose 6-phosphate, fructose and the like. It is known that the classification of the glycated hemoglobin differs between human and non-human mammals.

For example, human glycated hemoglobin is hemoglobin A wherein sugar is bonded to the amino group of N-terminal valine of β chain, from among human hemoglobins classified into hemoglobin A, hemoglobin F and hemoglobin A2, and is divided into Ala1, Ala2, A1b, A1c and the like. Of these, hemoglobin A1c, wherein D-glucose is non-enzymatically bonded to the amino group of N-terminal valine of β chain of hemoglobin A, accounts for most of human glycated hemoglobins.

Hemoglobin A1c is stable, and does not disappear until the life of blood erythrocyte (about 120 days) runs out, and therefore, can reflect an average blood glucose level of 1 to 3 months back. For diabetes mellitus patients, therefore, hemoglobin A1c is used as an important index of long-term blood glucose control.

In contrast, for rats, because the molecular kind of hemoglobin varies and the position of hemoglobin to which sugar bonds also varies, the classification of rat glycated hemoglobin is different from that of human glycated hemoglobin. In rats, hemoglobin A1 that accounts for a part of rat glycated hemoglobins is used as an index of long-term blood glucose control. For example, it has been reported that pioglitazone hydrochloride was administered to genetically obese and diabetic Wistar fatty rats and hemoglobin A1 value was measured 14 days later (EP-A 749751).

However, there is no report on a screening method for an agent for the treatment of human diabetes mellitus, which comprises selecting a compound that causes a decrease of not less than 0.5% in a glycated hemoglobin value in a diabetes mellitus model non-human mammal.

### DISCLOSURE OF THE INVENTION

There is a demand for a method capable of conveniently and precisely screening for an agent for the treatment of human diabetes mellitus, using a diabetes mellitus model non-human mammal.

The present inventors have conducted intensive studies in an attempt to solve the above-mentioned problems and first found that, by selecting a compound that causes a decrease of not less than 0.5% in a glycated hemoglobin value in a diabetes mellitus model non-human mammal, an agent for the treatment of human diabetes mellitus can be screened for. Based on this finding, the present inventors have further studied and completed the present invention.

Accordingly, the present invention relates to
1) a screening method for an agent for the treatment of human diabetes mellitus, which comprises administering a test compound to a diabetes mellitus model non-human mammal, and selecting a compound that causes a decrease of not less than 0.5% in a glycated hemoglobin value after administration;
2) the screening method of the above-mentioned 1), wherein the non-human mammal is a rodent;
3) the screening method of the above-mentioned 2), wherein the rodent is a rat;
4) the screening method of the above-mentioned 1), wherein the diabetes mellitus model non-human mammal is a Wistar fatty rat;
5) the screening method of the above-mentioned 1), wherein the diabetes mellitus model non-human mammal is a Zucker diabetic fatty rat;
6) the screening method of the above-mentioned 1), wherein after administration is 4 weeks after administration;
7) the screening method of the above-mentioned 1), wherein the agent for the treatment of human diabetes mellitus is a compound that causes a decrease of not less than 0.5% in a hemoglobin A1c value at 12 weeks after administration;
8) an agent for the treatment of human diabetes mellitus, which is obtained by the screening method of the above-mentioned 1); and the like.

The screening method of the present invention comprises administering a test compound to a diabetes mellitus model non-human mammal, and selecting a compound that causes a decrease of not less than 0.5% in a glycated hemoglobin value after administration.

By the “diabetes mellitus model non-human mammal” is meant a non-human mammal congenitally affected by diabetes mellitus or a non-human mammal that acquired diabetes mellitus.

As used herein, diabetes mellitus means a state of higher blood glucose level than healthy animal during satiation or fasting.

As a non-human mammal, mouse, rat, guinea pig, hamster, rabbit, dog, monkey and the like can be mentioned. Of these, rodents such as mouse, rat and the like are preferable, and rat is more preferable.

Preferable examples of the “diabetes mellitus model non-human mammal” include non-human mammals congenitally affected by diabetes mellitus such as db/db mouse, ob/ob mouse, KKA^{y} mouse, NOD mouse, NSY mouse, KK mouse, Wistar fatty rat, Zucker diabetic fatty (ZDF) rat, GK rat, OLETF rat, BB rat, WBN/Kob rat, Zucker fatty rat and the like; non-human mammals that acquired diabetes mellitus such as SD (Sprague-Dawley) rat and the like wherein diabetes mellitus has been induced by administration of alloxan or streptozotocin (STZ); and the like.

The “diabetes mellitus model non-human mammal” preferably includes Wistar fatty rat, Zucker diabetic fatty rat and the like, more preferably Wistar fatty rat.

While the “diabetes mellitus model non-human mammal” is commercially available, it can be also prepared by a method known per se.

In addition, the “diabetes mellitus model non-human mammal” is preferably one that stably shows high blood glucose level, and is not in a serious state of diabetes mellitus.

For example, when db/db mouse, ob/ob mouse, KKA^{y} mouse, Zucker diabetic fatty rat, GK rat, Wistar fatty rat and the like are used as the “diabetes mellitus model non-human mammal”, those at about 10 to about 30 weeks of age are preferably used.

In the screening method of the present invention, an impaired glucose tolerance model non-human mammal may be used as a diabetes mellitus model non-human mammal.

The “impaired glucose tolerance model non-human mammal” means a non-human mammal congenitally affected by impaired glucose tolerance or a non-human mammal that acquired impaired glucose tolerance.

As used herein, by the impaired glucose tolerance is meant a state where the blood glucose level is normal during fasting, but the blood glucose level once increased by glucose loading (e.g., after eating or during oral glucose tolerance test) does not decrease as compared with healthy animals even after the lapse of 1-2 hr. As the non-human mammal, those similar to the aforementioned can be used.

Preferable examples of the “impaired glucose tolerance model non-human mammal” include those similar to the “diabetes mellitus model non-human mammal” mentioned above. Of these, an animal showing a casual blood glucose level which is at a normal level or near the normal level, such as KK mouse, Zucker fatty rat and the like, is preferable.

In addition, as the “impaired glucose tolerance model non-human mammal”, from the mammals widely used as the aforementioned “diabetes mellitus model non-human mammal”, a mammal that has-not developed diabetes mellitus and of a comparatively young age, such as KKA^{y} mouse, Wistar fatty rat and the like, which are each about 4 to about 7 weeks of age, is also preferable.

The “test compound” may be any of, for example, peptide, protein, non-peptidic compound, synthetic compound, fermentation product, cell extract, plant extract, animal tissue extract and the like, which may be any of a known substance and a novel substance.

The method of administering a “test compound” to a “diabetes mellitus model non-human mammal” is not particularly limited. As the method of administering a test compound, for example, 1) a method comprising dissolving or suspending a test compound in water and the like as necessary and then orally administering same to an animal, 2) a method comprising mixing a test compound and a feed and then orally administering same to an animal, 3) a method comprising administering a test compound to an animal as an injection (e.g., subcutaneous injection, intravenous injection, intramuscular injection, intraperitoneal injection and the like), and the like can be mentioned.

While the dose of the “test compound” to a “diabetes mellitus model non-human mammal” varies depending on the administration subject, administration route and the like, when the “test compound” is orally administered to a mouse or a rat, for example, the dose per administration is generally about 0.0001-10000 mg/kg body weight, preferably 0.001-1000 mg/kg body weight, more preferably 0.01-100 mg/kg body weight.

While the administration frequency of the “test compound” to a “diabetes mellitus model non-human mammal” is not particularly limited, for example, about 1 to 3 times a day is preferable. The administration of a “test compound” to a “diabetes mellitus model non-human mammal” is preferably performed about 1 to 3 times a day, continuously until the measurement of a decrease in the glycated hemoglobin value.

The “glycated hemoglobin value” in the “decrease in the glycated hemoglobin value” means the proportion (%) of the amount of glycated hemoglobin to the total hemoglobin amount in the blood taken from a non-human mammal.

The “decrease in the glycated hemoglobin value” is a value that can be calculated from the following formula;
“decrease in the glycated hemoglobin value”=(“glycated hemoglobin value before test compound administration”−“glycated hemoglobin value after test compound administration”)−“decrease in the glycated hemoglobin value in test compound non-administration group”

The above-mentioned “decrease in the glycated hemoglobin value in test compound non-administration group” means an amount of decrease in the glycated hemoglobin value in a test compound non-administration group that is generally used as a control group, which value varied during the period of before and after the test compound administration in a test compound administration group.

The “glycated hemoglobin” used in the present invention means any hemoglobin to which a sugar such as D-glucose, glucose 6-phosphate, fructose and the like has bonded.

The “glycated hemoglobin value” of a diabetes mellitus model non-human mammal can be measured by, for example, a known method, such as affinity column chromatography and the like. The “glycated hemoglobin value” can be specifically measured with a glycohemoglobin analyzer [e.g., HLC-723GHbV A1c2.2 (product name)(manufactured by Tosoh Corporation)] for animals using an aminophenyl boronate affinity column.

For measurement of a “glycated hemoglobin value”, it is preferable to add an effective amount of an anticoagulant agent to the blood taken from a non-human mammal for the purpose of obtaining higher precision measurement values. Here, as the anticoagulant agent, for example, heparin, ethylenediaminetetraacetic acid (EDTA) and the like can be mentioned. For example, when heparin (e.g., physiological saline solution of heparin sodium) is used as an anticoagulant agent, generally, it is preferably used such that the heparin concentration in blood becomes about 10-50 U/ml or 0.1-0.5 mg/ml. When EDTA is used as an anticoagulant agent, generally, EDTA is preferably used as an aqueous solution so that the EDTA concentration in blood becomes about 0.005-0.02M.

The measurement time for a decrease in the glycated hemoglobin value is appropriately determined in consideration of the lifetime of hemoglobin in a diabetes mellitus model non-human mammal. As a specific measurement time, “not less than 4 weeks' lapse from test compound administration”, more specifically, “4 weeks after test compound administration”, “8 weeks after test compound administration”, “12 weeks after test compound administration” and the like can be mentioned.

However, when a decrease in the glycated hemoglobin value is observed shortly after administering a test compound to a diabetes mellitus model non-human mammal, the decrease in the glycated hemoglobin value may be measured, for example, after lapse of a short time such as “not less than 2 weeks after test compound administration” and the like.

The “compound that causes a decrease of not less than 0.5% in a glycated hemoglobin value” is more preferably “a compound that causes a decrease of not less than 1% in the glycated hemoglobin value”.

With regard to the “agent for the treatment of human diabetes mellitus” obtained by the screening method of the present invention, the following report exists on the evaluation criteria of human diabetes mellitus.

According to the report by the Japan Diabetes Society in 1999, diabetes mellitus is a condition wherein the fasting blood glucose level (glucose concentration in venous plasma) is not less than 126 mg/dl, the 2-hour value (glucose concentration in venous plasma) of the 75 g oral glucose tolerance test (75 g OGTT) is not less than 200 mg/dl or the casual blood glucose level (glucose concentration in venous plasma) is not less than 200 mg/dl. In addition, a condition that does not fall within the scope of the above definition of diabetes mellitus, and which is not a “condition wherein the fasting blood glucose level (glucose concentration in venous plasma) is less than 110 mg/dl or the 2-hour value (glucose concentration in venous plasma) of the 75 g oral glucose tolerance test (75 g OGTT) is less than 140 mg/dl” (normal type), is called the “borderline type”.

According to the reports by ADA (American Diabetes Association) in 1997 and by WHO in 1998, diabetes mellitus is a condition where the fasting blood glucose level (glucose concentration in venous plasma) is not less than 126 mg/dl, and the 2-hour value (glucose concentration in venous plasma) of the 75 g oral glucose tolerance test is not less than 200 mg/dl. In addition, according to the above reports, a condition where the fasting blood glucose level (glucose concentration in venous plasma) is less than 126 mg/dl, and the 2-hour value (glucose concentration in venous plasma) of the 75 g oral glucose tolerance test is not less than 140 mg/dl and less than 200 mg/dl is called impaired glucose tolerance. Furthermore, according to the ADA report, a condition where the fasting blood glucose level (glucose concentration in venous plasma) is not less than 110 mg/dl and less than 126 mg/dl, is called IFG (Impaired Fasting Glucose). On the other hand, according to the WHO report, a condition of IFG (Impaired Fasting Glucose) as such, where the 2-hour value (glucose concentration in venous plasma) of the 75 g oral glucose tolerance test is less than 140 mg/dl, is called IFG (Impaired Fasting Glycemia).

The “agent for the treatment of human diabetes mellitus” is a pharmaceutical agent that can treat at least one kind of the above-mentioned “diabetes mellitus”, “borderline type”, “impaired glucose tolerance”, “IFG (Impaired Fasting Glucose)” and “IFG (Impaired Fasting Glycemia)”. In addition, the “agent for the treatment of human diabetes mellitus” may be a pharmaceutical agent that prevents progression from the above-mentioned “borderline type”, “impaired glucose tolerance”, “IFG (Impaired Fasting Glucose)” or “IFG (Impaired Fasting Glycemia)” to “diabetes mellitus”.

The action mechanism of the “agent for the treatment of human diabetes mellitus” obtained by the screening method of the present invention is not particularly limited, and as the kind of the “agent for the treatment of human diabetes mellitus”, for example, insulin sensitizers, PPARγ agonists, PPARγ antagonists, PPARγ/α dual agonists, PPARγ/δ dual agonists, biguanides, insulin secretagogues, α-glucosidase inhibitors, adrenaline β3 agonists, insulins and derivatives thereof, GLP-1 receptor agonists, amylin agonists, phosphotyrosine phosphatase inhibitors, dipeptidylpeptidase IV inhibitors, glycogen phosphorylase inhibitors, glucose 6-phosphatase inhibitors, glucagon antagonists, somatostatin receptor agonists, SGLT (sodium-glucose cotransporter) inhibitors and the like can be mentioned.

As the “agent for the treatment of human diabetes mellitus”, for example, “a compound that causes a decrease of not less than 0.1% in a hemoglobin Alc value in human diabetes mellitus patients at 4 weeks after administration”, “a compound that causes a decrease of not less than 0.2% in a hemoglobin A1c value in human diabetes mellitus patients at 8 weeks after administration”, “a compound that causes a decrease of not less than 0.5% in a hemoglobin Alc value in human diabetes mellitus patients at 12 weeks after administration”, “a compound that causes a decrease of not less than 1% in a hemoglobin Alc value in human diabetes mellitus patients at 12 weeks after administration” and the like can be mentioned.

As used herein, the “hemoglobin A1c value” of “a decrease in a hemoglobin A1c value” means the proportion (%) of the amount of hemoglobin A1c to the total hemoglobin amount in the blood taken from a human diabetes mellitus patient. In addition, the “decrease in hemoglobin A1c value” means a value calculated from the following formula:
“decrease in hemoglobin A1c value (%)”=(“hemoglobin A1c value before test compound administration”−“hemoglobin A1c value after test compound administration”)−“decrease in hemoglobin A1c value in test compound non-administration group”

The above-mentioned “decrease in hemoglobin A1c value in test compound non-administration group” means an amount of decrease in the hemoglobin A1c value in a test compound non-administration group that is generally used as a control group, which value varied during the period of before and after the test compound administration in a test compound administration group.

The “hemoglobin A1c value” of a human diabetes mellitus patient can be measured by, for example, a known method, such as ion exchange column chromatography, affinity column chromatography and the like. The “hemoglobin A1c value” can be specifically measured with a glycohemoglobin analyzer [e.g., HLC-723GHbV A1c2.2 (product name) (manufactured by Tosoh Corporation); or CLC385 (product name)(manufactured by Primus Corporation, US)].

The “agent for the treatment of human diabetes mellitus” is preferably “a compound that causes a decrease of not less than 0.5% in a hemoglobin Alc value in human diabetes mellitus patients at 12 weeks after administration”, more preferably “a compound that causes a decrease of not less than 1% in a hemoglobin A1c value in human diabetes mellitus patients at 12 weeks after administration”. Here, “a decrease in a hemoglobin A1c value in human diabetes mellitus patients at 12 weeks after administration” preferably means “a decrease in hemoglobin A1c value after continuously administering the compound to human diabetes mellitus patients about 1 to 3 times a day for the period of 12 weeks”.

The “agent for the treatment of human diabetes mellitus” obtained by the screening method of the present invention can be administered to human diabetes mellitus patients as it is, or after being mixed with a pharmacologically acceptable carrier and the like to give a pharmaceutical composition.

Here, the pharmacologically acceptable carriers are exemplified by various organic or inorganic carrier substances in common use as materials for pharmaceutical preparations, and they are formulated as excipients, lubricants, binders, and disintegrants for solid preparations; solvents, solubilizers, suspending agents, isotonizing agents, buffers, soothing agents for liquid preparations; and the like. In addition, other additives for pharmaceutical preparations, such as antiseptics, antioxidants, coloring agents, and sweetening agents, may be also used as necessary.

Preferable examples of the excipients include lactose, saccharose, D-mannitol, D-sorbitol, starch, gelatinized starch, dextrin, crystalline cellulose, low-substituted hydroxypropylcellulose, carboxymethylcellulose sodium, gum arabic, dextrin, pullulan, light silicic anhydride, synthetic aluminum silicate, magnesium metasilicate aluminate and the like.

Preferable examples of the lubricants include magnesium stearate, calcium stearate, talc, colloidal silica and the like.

Preferable examples of the binders include gelatinized starch, sucrose, gelatin, gum arabic, methylcellulose, carboxymethylcellulose, carboxymethylcellulose sodium, crystalline cellulose, saccharose, D-mannitol, trehalose, dextrin, pullulan, hydroxypropylcellulose, hydroxypropylmethylcellulose, polyvinylpyrrolidone and the like.

Preferable examples of the disintegrants include lactose, saccharose, starch, carboxymethylcellulose, carboxymethylcellulose calcium, croscarmellose sodium, carboxymethyl starch sodium, light silicic anhydride, low-substituted hydroxypropylcellulose and the like.

Preferable examples of the solvents include water for injection, physiological saline, Ringer's solution, alcohol, propylene glycol, polyethylene glycol, sesame oil, corn oil, olive oil, cottonseed oil and the like.

Preferable examples of the solubilizers include polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like.

Preferable examples of the suspending agents include surfactants such as stearyltriethanolamine, sodium lauryl sulfate, laurylaminopropionic acid, lecithin, benzalkonium chloride, benzethonium chloride, monostearic glycerol and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose and the like; polysorbates, polyoxyethylene hydrogenated castor oil and the like.

Preferable examples of the isotonizing agents include sodium chloride, glycerol, D-mannitol, D-sorbitol, glucose and the like.

Preferable examples of the buffers include buffer solutions of phosphates, acetates, carbonates, citrates and the like.

Preferable examples of the soothing agents include benzyl alcohol and the like.

Preferable examples of the antiseptics include p-oxybenzoic acid esters, chlorobutanol, benzyl alcohol, phenethyl alcohol, dehydroacetic acid, sorbic acid and the like.

Preferable examples of the antioxidants include sulfites, ascorbates and the like.

Preferable examples of the coloring agents include water-soluble tar-colors for food (e.g., food colors such as Food Color Red Nos. 2 and 3, Food Color Yellow Nos. 4 and 5, Food Color Blue Nos. 1 and 2 and the like), water-insoluble lake colors (e.g., aluminum salts of the aforementioned water-soluble tar colors for food and the like), natural colors (e.g., β-carotene, chlorophyll, red iron oxide and the like), and the like.

Preferable examples of the sweetening agents include saccharin sodium, dipotassium glycyrrhetinate, aspartame, stevia and the like.

Examples of the dosage forms of the pharmaceutical composition include oral preparations such as tablets (including sublingual tablets and orally disintegrating tablets), capsules (including soft capsules and microcapsules), granules, powders, troches, syrups, emulsions, suspensions and the like; and parenteral preparations such as injections (e.g., subcutaneous injections, intravenous injections, intramuscular injections, intraperitoneal injections, drip infusions and the like), external preparations (e.g., transdermal preparations, ointments and the like), suppositories (e.g., rectal suppositories, vaginal suppositories and the like), pellets, preparations for nasal administration, transpulmonary agents (inhalant), eye drops and the like. Each of these preparations can be orally or parenterally administered safely.

These preparations may be a controlled-release preparation such as a rapid release preparation, a sustained-release preparation and the like (e.g., sustained-release microcapsules and the like).

The pharmaceutical composition can be prepared according to conventional methods in the fields of pharmaceutical manufacturing techniques, such as the methods described in the Japanese Pharmacopoeia and the like. Specific production methods for the preparations are hereinafter described in detail.

While the content of the “agent for the treatment of human diabetes mellitus” in a pharmaceutical composition varies depending on the dosage form, dose of the “agent for the treatment of human diabetes mellitus” and the like, it is, for example, about 0.01-100 wt %.

For example, an oral preparation is produced by adding, to the active ingredient, an excipient (e.g., lactose, saccharose, starch, D-mannitol and the like), a disintegrant (e.g., carboxymethylcellulose calcium and the like), a binder (e.g., gelatinized starch, gum arabic, carboxymethylcellulose, hydroxypropylcellulose, polyvinylpyrrolidone and the like) or a lubricant (e.g., talc, magnesium stearate, polyethylene glycol 6000 and the like) and the like, compression molding the obtained mixture, and, if necessary, coating by a method known per se using a coating base for the purpose of taste masking, enteric coating or sustained release.

Examples of the coating base include a sugar coating base, a water-soluble film coating base, an enteric film coating base, a sustained-release film coating base and the like.

As the sugar coating base, saccharose is employed. Further, one or more kinds selected from talc, precipitated calcium carbonate, gelatin, gum arabic, pullulan, carnauba wax and the like may be used in combination.

Examples of the water-soluble film coating base include cellulose polymers such as hydroxypropylcellulose, hydroxypropylmethylcellulose, hydroxyethylcellulose, methylhydroxyethylcellulose; synthetic polymers such as polyvinylacetal diethylaminoacetate, aminoalkyl methacrylate copolymer E [Eudragit E (trademark), Rhom Pharma] and polyvinylpyrrolidone; and polysaccharides such as pullulan, and the like.

Examples of the enteric film coating base include cellulose polymers such as hydroxypropylmethylcellulose phthalate, hydroxypropylmethylcellulose acetate succinate, carboxymethylethylcellulose, cellulose acetate phthalate; acrylic acid polymers such as methacrylic acid copolymer L [Eudragit L (trademark), Rhom Pharma], methacrylic acid copolymer LD [Eudragit L-30D55 (trademark), Rhom Pharma], methacrylic acid copolymer S [Eudragit S (trademark), Rhom Pharma]; natural products such as shellac, and the like.

Examples of the sustained-release film coating base include cellulose polymers such as ethylcellulose; acrylic acid polymers such as aminoalkyl methacrylate copolymer RS [Eudragit RS (trademark), Rhom Pharma] and ethyl acrylate-methyl methacrylate copolymer suspension [Eudragit NE (trademark), Rhom Pharma] and the like.

Two or more of the above-mentioned coating bases may be used in admixture at an appropriate ratio. For coating, a shading agent such as titanium oxide, red ferric oxide and the like may be used.

Injections are produced by dissolving, suspending or emulsifying the active ingredient in an aqueous solvent (e.g., distilled water, physiological saline, Ringer's solution and the like) or an oleaginous solvent (e.g., vegetable oils such as olive oil, sesame oil, cotton seed oil, corn oil; propylene glycol, and the like) and the like, together with a dispersant (e.g., polysorbate 80, polyoxyethylene hydrogenated castor oil 60, polyethylene glycol, carboxymethylcellulose, sodium alginate and the like), a preservative (e.g., methylparaben, propylparaben, benzyl alcohol, chlorobutanol, phenol and the like), an isotonizing agent (e.g., sodium chloride, glycerol, D-mannitol, D-sorbitol, glucose and the like) and the like. On such occasions, if desirable, additives such as a solubilizer (e.g., sodium salicylate, sodium acetate and the like), a stabilizer (e.g., human serum albumin and the like), a soothing agent (e.g., benzyl alcohol and the like) and the like, may be used.

While the dose of the “agent for the treatment of human diabetes mellitus” obtained by the screening method of the present invention varies depending on the administration subject, administration route, conditions and the like, for oral administration to an adult diabetes mellitus patient (body weight 60 kg), for example, the dose per administration is about 0.001 to 10000 mg, preferably 0.01 to 1000 mg, more preferably 0.1 to 500 mg, which dose is desirably administered 1 to 3 times a day.

The “agent for the treatment of human diabetes mellitus” obtained by the screening method of the present invention can be used in combination with a drug such as a therapeutic agent for diabetes mellitus, a therapeutic agent for diabetic complications, an antihyperlipidemic agent, a hypotensive agent, an antiobesity agent, a diuretic agent, a chemotherapeutic agent, an immunotherapeutic agent, antithrombotic agent, a therapeutic agent for osteoporosis, an antidementia agent, an erectile dysfunction improving agent, a therapeutic agent for incontinence or pollakiuria and the like (hereinafter to be abbreviated as a concomitant drug), for the purpose of enhancing its diabetes mellitus treatment effect, reducing the dose, or reducing the side effects and the like.

On such occasions, the timing of administration of the “agent for the treatment of human diabetes mellitus” and that of the concomitant drug are not limited. They may be administered simultaneously or in a staggered manner to the administration subject. Moreover, the “agent for the treatment of human diabetes mellitus” and the concomitant drug may be administered as-two kinds of preparations, each containing its own active ingredient, or may be administered as a single preparation containing both active ingredients.

The dose of the concomitant drug can be appropriately selected based on a clinically employed dose. The proportion of the “agent for the treatment of human diabetes mellitus” and the concomitant drug can be appropriately determined according to the administration subject, the administration route, the clinical conditions, the combination, and other factors. In cases where the administration subject is a human, for instance, the concomitant drug may be used in an amount of 0.01 to 100 parts by weight per part by weight of the “agent for the treatment of human diabetes mellitus”.

Examples of the therapeutic agent for diabetes mellitus include insulin preparations (e.g., animal insulin preparations extracted from the bovine or swine pancreas; human insulin preparations synthesized by a genetic engineering technique using Escherichia coli or a yeast; zinc insulin; protamine zinc insulin; fragment of insulin or derivatives thereof (e.g., INS-1 and the like), and the like), insulin sensitizers [e.g., pioglitazone hydrochloride, rosiglitazone (maleate), GI-262570, JTT-501, MCC-555, YM-440, KRP-297, CS-011, FK-614, NN-622, AZ-242, BMS-298585, EML-16336, compounds described in WO 99/58510 (e.g., (E)-4-[4-(5-methyl-2-phenyl-4-oxazolylmethoxy)benzyloxyimino]-4-phenylbutyric acid) and the like], PPARγ agonists, PPARγ antagonists, PPARγ/α dual agonists, α-glucosidase inhibitors (e.g., voglibose, acarbose, miglitol, emiglitate and the like), biguanides (e.g., phenformin, metformin, buformin and the like), insulin secretagogues [sulfonylureas (e.g., tolbutamide, glibenclamide, gliclazide, chlorpropamide, tolazamide, acetohexamide, glyclopyramide, glimepiride, glipizide, glybuzole and the like), repaglinide, senaglinide, nateglinide, mitiglinide or its calcium salt hydrate], GLP-1 receptor agonists [e.g., GLP-1, NN-2211, AC-2993(exendin-4), BIM-51077, Aib(8,35)hGLP-1(7,37)NH₂ and the like], amylin agonists (e.g., pramlintide and the like), phosphotyrosine phosphatase inhibitors (e.g., vanadic acid and the like), dipeptidylpeptidase IV inhibitors (e.g., NVP-DPP-278, PT-100, P32/98, LAF-237 and the like), β3 agonists (e.g., CL-316243, SR-58611-A, UL-TG-307, SB-226552, AJ-9677, BMS-196085, AZ40140 and the like), gluconeogenesis inhibitors (e.g., glycogen phosphorylase inhibitors, glucose-6-phosphatase inhibitors, glucagon antagonists, somatostatin receptor agonists and the like), SGLT (sodium-glucose cotransporter) inhibitors (e.g., T-1095 and the like) and the like.

Examples of the therapeutic agent for diabetic complications include aldose reductase inhibitors (e.g., tolrestat, epalrestat, zenarestat, zopolrestat, minalrestat, fidarestat, SNK-860, CT-112 and the like), neurotrophic factors and a promoting agent thereof (e.g., NGF, NT-3, BDNF, neurotrophin production-secretion promoters described in WO 01/14372 (e.g., 4-(4-chlorophenyl)-2-(2-methyl-1-imidazolyl)-5-[3-(2-methylphenoxy)propyl]oxazole and the like) and the like), neuronegenesis promoters (e.g., Y-128 and the like), PKC inhibitors (e.g., LY-333531 and the like), AGE inhibitors (e.g., ALT946, pimagedine, pyratoxathine, N-phenacylthiazolium bromide (ALT766), EXO-226 and the like), reactive oxygen scavengers (e.g., thioctic acid and the like), and cerebral vasodilators (e.g., tiapuride, mexiletine, and the like).

As the antihyperlipidemic agent, for example, statin compounds (e.g., cerivastatin, pravastatin, simvastatin, lovastatin, atorvastatin, fluvastatin, itavastatin or salts thereof (e.g., sodium salt and the like) and the like), which are cholesterol synthetase inhibitors, squalene synthetase inhibitors (e.g., compounds described in WO 97/10224, such as N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid and the like) and fibrate compounds having triglyceride lowering action (e.g., bezafibrate, clofibrate, simfibrate, clinofibrate and the like), ACAT inhibitors (e.g., Avasimibe, Eflucimibe and the like), anion exchange resins (e.g., cholestyramine and the like), probucol, nicotinic acid drugs (e.g., nicomol, niceritrol and the like), ethyl eicosapentaenoate, vegetable sterol (e.g., soysterol, yoryzanol and the like) and the like can be mentioned.

Examples of the hypotensive agent include angiotensin converting enzyme inhibitors (e.g., captopril, enalapril, delapril, and the like), angiotensin II antagonists (e.g., candesartan cilexetil, losartan, eprosartan, valsantan, termisartan, irbesartan, tasosartan, and the like), calcium antagonists (e.g., manidipine, nifedipine, amlodipine, efonidipine, nicardipine, and the like), potassium channel openers (e.g., levcromakalim, L-27152, AL 0671, NIP-121, and the like), clonidine, and the like.

Examples of the antiobesity agent include antiobesity drugs acting on the central nervous system (e.g., dexfenfluramine, fenfluramine, phentermine, sibutramine, anfepramone, dexamphetamine, mazindol, phenylpropanolamine, clobenzorex and the like), pancreatic lipase inhibitors (e.g., orlistat and the like), β3 agonists (e.g., CL-316243, SR-58611-A, UL-TG-307, SB-226552, AJ-9677, BMS-196085, AZ40140 and the like), anorectic peptides (e.g., leptin, CNTF (Ciliary Neurotrophic Factor) and the like), cholecystokinin agonists (e.g., lintitript, FPL-15849 and the like), and the like.

Examples of the diuretic agent include xanthine derivatives (e.g., theobromine and sodium salicylate, theobromine and calcium salicylate and the like), thiazide preparations (e.g., ethiazide, cyclopenthiazide, trichlormethiazide, hydrochlorothiazide, hydroflumethiazide, benzylhydrochlorothiazide, penflutizide, polythiazide, methyclothiazide and the like), antialdosterone preparations (e.g., spironolactone, triamterene and the like), carbonic anhydrase inhibitors (e.g., acetazolamide and the like), chlorobenzenesulfonamide preparations (e.g., chlorthalidone, mefruside, indapamide and the like), azosemide, isosorbide, ethacrynic acid, piretanide, bumetanide, furosemide and the like.

Examples of the chemotherapeutic agent include alkylating agents (e.g., cyclophosphamide, ifosphamide and the like), metabolic antagonists (e.g., methotrexate, 5-fluorouracil and a derivative thereof and the like), antitumor antibiotics (e.g., mitomycin, adriamycin and the like), plant-derived antitumor agents (e.g., vincristine, vindesine, taxol and the like), cisplatin, carboplatin, etopoxide and the like. Of these, 5-fluorouracil derivatives such as Furtulon and Neo-Furtulon and the like are preferable.

Examples of the immunotherapeutic agent include microorganism- or bacterium-derived components (e.g., muramyl dipeptide derivatives, Picibanil and the like), immunopotentiator polysaccharides (e.g., lentinan, schizophyllan, krestin and the like), genetically engineered cytokines (e.g., interferons, interleukins (IL) and the like), colony stimulating factors (e.g., granulocyte colony stimulating factor, erythropoietin and the like) and the like. Of these, interleukins such as IL-1, IL-2, IL-12 and the like are preferable.

As the antithrombotic agent, for example, heparin (e.g., heparin sodium, heparin calcium, dalteparin sodium and the like), warfarin (e.g., warfarin potassium and the like), antithrombins (e.g., argatroban and the like), thrombolytic agents (e.g., urokinase, tisokinase, alteplase, nateplase, monteplase, pamiteplase and the like), platelet aggregation inhibitors (e.g., ticlopidine hydrochloride, cilostazol, ethyl eicosapentaenoate, beraprost sodium, sarpogrelate hydrochloride and the like) and the like can be mentioned.

As the therapeutic agent for osteoporosis, for example, alfacalcidol, calcitriol, elcatonin, calcitonin salmon, estriol, ipriflavone, pamidronate disodium, alendronate sodium hydrate, incadronate disodium and the like can be mentioned.

As the antidementia agent, for example, tacrine, donepezil, rivastigmine, galanthamine and the like can be mentioned.

As the erectile dysfunction improving agent, for example, apomorphine, sildenafil citrate and the like can be mentioned.

As the therapeutic agent for incontinence or pollakiuria, for example, flavoxate hydrochloride, oxybutynin hydrochloride, propiverine hydrochloride and the like can be mentioned.

Furthermore, drugs confirmed to have a cachexia improvement action in animal models and clinical situations, such as cyclooxygenase inhibitors (e.g., indomethacin and the like) (Cancer Research, vol. 49, pp. 5935-5939, 1989), progesterone derivatives (e.g., megestrol acetate) (Journal of Clinical Oncology, vol. 12, pp. 213-225, 1994), glucosteroids (e.g., dexamethasone and the like), metoclopramide pharmaceuticals, tetrahydrocannabinol pharmaceuticals (the above references are applied to both), fat metabolism ameliorating agents (e.g., eicosapentaenoic acid and the like) (British Journal of Cancer, vol. 68, pp. 314-318, 1993), growth hormones, IGF-1, and antibodies to the cachexia-inducing factor TNF-α, LIF, IL-6 or oncostatin M, and the like can be mentioned as the concomitant drug.

The present invention is explained in more detail in the following by referring to Experimental Examples, which are not to be construed as limitative.

### EXAMPLES

### Experimental Example 1

The 19-week-old male Wistar fatty rats (21 rats), that showed a blood glucose level [mean i standard error measured by Hitachi 7070 (product name, manufactured by Hitachi, Ltd.)] of 382.7±33.9 mg/dl, and a glycated hemoglobin value [mean±standard error] of 7.8±0.4%, were divided into 3 groups (7 per group) of a, b and c.

In this Experimental Example, the blood of the rats were taken in a microtube finally containing 0.01M EDTA as an anticoagulant agent, for the measurement of blood glucose level and glycated hemoglobin value.

The glycated hemoglobin value was measured by high performance liquid chromatograph (HLC-723GhbVA1c2.2 (product name, manufactured by Tosoh Corporation)) using an aminophenyl boronate affinity column and an absorbance (415 nm) detection method. For the mobile phase, eluents A, B (manufactured by Tosoh Corporation) were used, and the conditions set for chromatography, such as column temperature, flow rate and the like, followed the standard conditions set for this chromatography.

The aforementioned a group was given a general powder diet (CLEA Japan, Inc., CE-2), b group and c group were given the aforementioned powder diet containing pioglitazone hydrochloride in a daily amount of 0.8 mg/kg body weight and 2.4 mg/kg body weight, respectively (weight ratio to diet of 0.0015% and 0.0045%, respectively), and reared for 5 weeks. The glycated hemoglobin value was measured at 4 and 5 weeks after the start of the experiment.

As a result, in the control a group, glycated hemoglobin value decreased by 0.3% and 0.6%, respectively at 4 and 5 weeks after the start of the experiment. In the b group, glycated hemoglobin value decreased by 1.4% and 1.9%, respectively at 4 and 5 weeks after the start of the administration. In the c group, moreover, glycated hemoglobin value decreased by 2.8% and 3.3%, respectively at 4 and 5 weeks after the start of the administration. That is, the decrease in the glycated hemoglobin value in b group at 4 and 5 weeks after the start of the administration was 1.1% (1.4%-0.3%) and 1.3% (1.9%-0.6%), respectively. In addition, the decrease in the glycated hemoglobin value in c group at 4 and 5 weeks after the start of the administration was 2.5% (2.8%-0.3%) and 2.7% (3.3%-0.6%), respectively.

### Experimental Example 2

Diabetes mellitus patients (134 patients, age 57.6±11.5 years old, fasting blood glucose level 183.2±35.2 mg/dl, hemoglobin A1c value 9.27±1.60%) were divided into 2 groups. Placebos were given to one group (66 patients) and pioglitazone hydrochloride (30 mg daily dose) was orally administered to the other group (68 patients) for 12 weeks, and the hemoglobin A1c value was measured at 8 and 12 weeks after start of the administration.

As a result, the placebo administration group showed a decrease by 0.02% in the hemoglobin A1c value at 8 and 12 weeks after the start of the administration. In addition, the pioglitazone hydrochloride administration group showed a decrease by 0.70% and 1.08%, respectively, in the hemoglobin A1c value at 8 and 12 weeks after the start of the administration. Namely, a decrease in hemoglobin A1c value in the pioglitazone hydrochloride administration group at 8 and 12 weeks after start of the administration was 0.68% (0.70%-0.02%) and 1.06% (1.08%-0.02%), respectively.

### Industrial Applicability

According to the screening method of the present invention, an agent for the treatment of human diabetes mellitus can be screened for conveniently and with high precision.

1. A screening method for an agent for the treatment of human diabetes mellitus, which comprises administering a test compound to a diabetes mellitus model non-human mammal, and selecting a compound that causes a decrease of not less than 0.5% in a glycated hemoglobin value after administration.

2. The screening method of claim 1, wherein the non-human mammal is a rodent.

3. The screening method of claim 2, wherein the rodent is a rat.

4. The screening method of claim 1, wherein the diabetes mellitus model non-human mammal is a Wistar fatty rat.

5. The screening method of claim 1, wherein the diabetes mellitus model non-human mammal is a Zucker diabetic fatty rat.

6. The screening method of claim 1, wherein after administration is 4 weeks after administration.

7. The screening method of claim 1, wherein the agent for the treatment of human diabetes mellitus is a compound that causes a decrease of not less than 0.5% in a hemoglobin A1c value at 12 weeks after administration.

8. An agent for the treatment of human diabetes mellitus, which is obtained by the screening method of claim 1.
